(19) United States
(12) Patent Application Publication
(10) Pub. No.: US 2010/0099719 A1
(43) Pub. Date: Apr. 22, 2010

(54) COMPOSITION AND METHOD FOR TREATING PROTEINURIA

(75) Inventor: Yuenian Eric SHI, Roslyn Heights, NY (US)

Correspondence Address: LAW OFFICES OF ALBERT WAI-KIT CHAN, PLLC, 141-07 20TH AVENUE, WORLD PLAZA, SUITE 604, WHITESTONE, NY, 11357, US

(73) Assignee: DIEREPHARMA, INC., Roslyn Heights, NY (US)

(21) Appl. No.: 12/582,925

(22) Filed: Oct. 21, 2009

Related U.S. Application Data
(60) Provisional application No. 61/196,990, filed on Oct. 21, 2008.

(51) Int. Cl. A61K 31/44

(52) U.S. Cl. 514/345

(57) ABSTRACT

The present invention discloses 5-methyl-1-(substituted phenyl)-2(1H)-pyridones having the ability to reduce proteinuria and for treating kidney diseases. The ability to reduce proteinuria has a renoprotective effect for slowing the progression of kidney diseases. A representative example of 5-methyl-(1-substituted phenyl)-2(1H)-pyridones is 1-(3′-fluorophenyl)-5-methyl-2(1H)-pyridone, AKF-PD. Accordingly, the present invention provides compositions comprising one or more compounds selected from the group consisting of 5-methyl-1-(substituted phenyl)-2(1H)-pyridones and methods of using the same to treat proteinuria and kidney diseases.

This application claims benefit of U.S. Ser. No. 61/196,990, filed Oct. 21, 2008, the entire content of which is incorporated by reference into this application.

Throughout this application, various references or publications are cited. Disclosures of these references or publications in their entireties are hereby incorporated by reference into this application in order to more fully describe the state of the art to which this invention pertains.

### FIELD OF THE INVENTION

This invention is related to compositions comprising 5-methyl-1-(substituted phenyl)-2(1H)-pyridones and methods of using the same to treat proteinuria. Since proteinuria is a major symptom in chronic kidney disease (CKD), treating proteinuria is expected to improve renal functions in CKD and to slow the progression of the disease.

### BACKGROUND OF THE INVENTION

Each year in the United States, more than 100,000 people are diagnosed with kidney failure. Kidney failure is the final stage of chronic kidney disease (CKD). Diabetes and hypertension are the two most common causes for kidney disease (United States Renal Data System. USRDS 2007 Annual Data Report.). The progression of CKD is a complex process involving various intricate intracellular signaling pathways. Inflammation is one determinant of the progression of CKD.

Kidney disease or damage that results as a complication of diabetes is called diabetic nephropathy, which is characterized by glomerular hyperfiltration, extracellular matrix accumulation, glomerular enlargement, mesangial expansion, and intertubular fibrosis, resulting ultimately in diabetic glomerulosclerosis and progressive renal failure. Early diagnosis of diabetes and early intervention are critical in slowing the progression towards renal failure seen in many type and a significant percentage of type 2 diabetics. Kidney failure originated from diabetes accounts for nearly 44 percent of new cases of kidney failure [United States Renal Data System. USRDS 2007 Annual Data Report. Bethesda, Md.: NIDDKD, NIH USDHHS; 2007]. Nearly 24 million people in the United States have diabetes [National Diabetes Statistics, 2007. Bethesda, Md.: NIDDKD, NIH USDHHS; 2008. Even when diabetes is controlled, the disease can lead to CKD and kidney failure. Around 20 to 30 percent of people with diabetes develop diabetic nephropathy, nearly 180,000 people eventually end up with kidney failure (United States Renal Data System. USRDS 2007 Annual Data Report).

Hypertension is another common cause for kidney disease (United States Renal Data System. USRDS 2007 Annual Data Report.) People with diabetes are prone to hypertension. There are many pharmaceutical research efforts aiming to aggressively slow down the progression of chronic kidney disease.

Chronic kidney disease has two important abnormal clinical test results: decreased GFR (Glomerular Filtration Rate) and high protein (albumin) content in the urine (proteinuria). For example, levels of urinary albumin excretion greater than normal are observed frequently in patients with type 2 diabetes. Diabetic kidney disease takes many years to develop. Over several years, people who are developing kidney disease will have small amounts of the blood protein albumin begin to leak into their urine. Moderately increased levels of albuminuria, so-called microalbuminuria, are predictive both of progressive renal function loss up to diabetic nephropathy, and of cardiovascular morbidity and mortality (Soldatos G. and Cooper M E. Diabetic nephropathy: important pathophysiologic mechanisms. Diabetes Res Clin Pract, 2008, 82 Suppl 1:S75-9; Araki S., et al: Clinical impact of reducing microalbuminuria in patients with type 2 diabetes mellitus, Diabetes Res Clin Pract, 2008, 82 Suppl 1:S54-8). As the disease progresses, more albumin and other proteins leak into the urine. This stage is termed as macroalbuminuria or proteinuria. As the amount of protein in the urine increases, the kidneys' filtering function usually begins to drop. As kidney damage develops, blood pressure often rises as well. Recent studies indicate that proteinuria can be seen not only as a result (disease marker) of kidney damage but also as a direct cause (poison) for kidney damage [de Zeeuw D. Semin. Nephrol. 2007 March, 27(2):172-81; Mauro Abbate, J. Am. Soc. Nephrol. 17: 2974-2984, 2006). Therefore, therapies to reduce proteinuria are a common clinical practice for slowing the progressing of kidney diseases [Venkat K. K., South Med. J., 2004, 97(10):969-979].

As aforementioned, there is growing evidence that proteinuria is not only a marker of disease progression but also a cause or risk factor in the progression of renal disease. There are evidences to indicate that renal injury from proteinuria occurs through multiple pathways, including induction of tubular chemokine expression and complement activation that lead to inflammatory cell infiltration in the interstitium and sustained fibrogenesis. Macrophages are prominent in the interstitial inflammatory infiltrate. This cell type mediates progression of renal injury to the extent that macrophage numbers in renal biopsy predict renal survival in patients with chronic renal disease. Chemoattractants and adhesive molecules for inflammatory cells are up-regulated by excess ultra-filtered protein load of proximal tubular cells via activation of NF κB-dependent and p38 pathways. (Protein Overload Induces Fractalkine Up-regulation in Proximal Tubular Cells through Nuclear Factor κB- and p38 Mitogen-Activated Protein Kinase-Dependent Pathways. J. Am. Soc. Nephrol. 14: 2436-2446, 2003). This mechanism is a potential target for therapeutic approaches, as shown by beneficial effects of inhibitory molecules of NF κB or p38 in experimental studies.

The p38 and TGF-β1/Smad signaling pathways play critical roles in inflammation and fibrogenesis, respectively. TGF-β1 plays a key role in renal fibrosis in both experimental and human kidney diseases (Border W A., et al: Suppression of experimental glomerulonephritis by antiserum against transforming growth factor beta 1, Nature 1990, 346:371-374; Peters H., et al: Transforming growth factor-beta in human glomerular injury, Curr. Opin. Nephrol. Hypertension 1997, 6:389-393). TGF-β1 binds to the constitutively active TGF-β type II receptor (TGF-RII), which in turn recruits, phosphorylates, and activates TGF-β type I receptor (TGFRI, ALK5). The active form of TGFRI then phosphorylates Smad2 and Smad3 to form a hetero-oligomeric complex with Smad4, which translocates into the nucleus to regulate transcription of target genes. Increased Smad2 and Smad3 activities have been observed in patients with diabetic nephropathy and glomerulonephritis as well as experimental models of renal disease (Wang W., et al: Transforming growth factor-beta and Smad signalling in kidney diseases. Nephrology 2005, 10:48-56). There is increasing evidence that blockade of TGF-β1 action can ameliorate renal fibrosis (Fukasawa H., et al: Treatment with anti-TGF-beta antibody ameliorates chronic progressive nephritis by inhibiting Smad/TGF-beta signaling, Kidney Int. 2004, 65:63-7422; Grygielko E. T., et al: Inhibition of gene markers of fibrosis with a novel inhibitor of transforming growth factor-beta type I receptor kinase in puromycin-induced nephritis. J. Pharmacol. Exp. Ther. 2005, 313:943-951, J. Pharmacol. Exp. Ther. 2005, 313:943-951). TGF-β1/Smad signaling pathways are central to the progression of renal fibrosis, and inhibition of the TGF-β1/Smad signaling pathway may offer a therapeutic treatment for renal fibrosis.

The role of the p38 MAPK signaling has been extensively studied and its involvement in inflammation has been established (Han J, Lee J. D., Bibbs L., Ulevitch R. J.: A MAP kinase targeted by endotoxin and hyperosmolarity in mammalian cells. Science 1994, 265:808-811). p38 is a member of intracellular mitogen-activated protein kinase (MAPK) that regulates many inflammatory cytokines. Activation of p38 MAPK can induce the production and secretion of proinflammatory cytokines such as IL-18 and TNF-α.

In turn, IL-18 and TNF-α can activate p38 MAPK, which leads to autocrine and paracrine promotion of an inflammatory response that exacerbates kidney injury (Geng Y., et al. J. Clin. Invest. 1996, 98:2425-2430; Nephrol. Dial. Transplant 2002, 17:399-407). Increased activity of p38 MAPK has been observed in patients suffering from inflammatory bowel disease, human diabetic nephropathy, and glomerulonephritis (Hommes D., et al: Inhibition of stress-activated MAP kinases induces clinical improvement in moderate to severe Crohn's disease, Gastroenterology 2002, 122:7-14; Adhikary L., et al: Abnormal p38 mitogen-activated protein kinase signaling in human and experimental diabetic nephropathy, Diabetologia 2004, 47:1210-1222; Stambe C., et al: p38 mitogen-activated protein kinase activation and cell localization in human glomerulonephritis: correlation with renal injury, J. Am. Soc. Nephrol. 2004, 15:326-336). p38 MAPK activation has been demonstrated in human and experimental diabetic nephropathy (Adhikary L., et al: Abnormal p38 mitogen-activated protein kinase signalling in human and experimental diabetic nephropathy, Diabetologia 2004, 47:1210-1222). The activation of p38 MAPK in intrinsic renal cells and infiltrating leukocytes has been found to correlate with renal dysfunction and histopathology in human glomerulonephritis (Stambe C., et al: p38 mitogen-activated protein kinase activation and cell localization in human glomerulonephritis: correlation with renal injury, J. Am. Soc. Nephrol. 2004, 15:326-336).

Recent successful development of anti-cytokine therapeutics including those that block TNF-α and IL-1, and more recently IL-6 has spurred research into identifying new cytokine-based target. Several anti-fibrotic or anti-cirrhotic agents have been developed over the past few years, and most of them exert their effects by indirectly inhibiting the p38 pathway (Wu L. M., et al: A novel synthetic oleanolic acid derivative (CPU-II2) attenuates liver fibrosis in mice through regulating the function of hepatic satellite cells, J. Biomed. Sci. 2008, 15:251-9; Hattori S, et al: FR-167653, a selective p38 MAPK inhibitor, exerts salutary effect on liver cirrhosis through down-regulation of Runx2, Laboratory Investigation 2007, 87:591-601). Investigation into the mechanisms of actions of most of these drugs showed that blockade of p38 MAPK pathway in the myofibroblasts was the key mechanism in amelioration of tissue fibrosis. Preclinical studies show that blockade of p38 MAPK with various p38 MAPK kinase inhibitors is efficacious in several disease models, including arthritis and other joint diseases, septic shock, myocardial injury, and kidney injury (Stambe C, et al: p38 mitogen-activated protein kinase activation and cell localization in human glomerulonephritis: correlation with renal injury J. Am. Soc. Nephrol. 2004, 15:326-336; Lee J. C., et al: p38 mitogen-activated protein kinase inhibitors—mechanisms and therapeutic potentials, Pharmacol. Ther. 2003, 82:389-397; Stambe C., et al: Blockade of p38alpha MAPK ameliorates acute inflammatory renal injury in rat anti-GBM glomerulonephritis, J. Am. Soc. Nephrol. 2003, 14:338-351). The interference of this pathway can ameliorate renal fibrosis in a rat model of unilateral ureteral obstruction and anti-glomerular basement membrane disease. Koshikawa and colleagues (Role of p38 mitogen-activated protein kinase activation in podocyte injury and proteinuria in experimental nephrotic syndrome. J. Am. Soc. Nephrol. 2005, 16:2690-2701) demonstrated that pretreatment with p38 MAPK inhibitor can reduce podocyte injury and proteinuria in puromycin-induced experimental nephrotic syndrome.

Currently, the drugs useful for reducing proteinuria in CKD are drugs used for treating hypertension. Two types of drugs, angiotensin-converting enzyme (ACE) inhibitors and angiotensin receptor blockers (ARBs), have proven effective in reducing proteinuria levels and slowing the progression of kidney disease. Although the exact pharmacological mechanisms of ACE/ARB are not clear, no data indicate they are involved in inflammatory processes. New agents with different mechanism are needed for proteinuria and CKD, particularly for diabetic related proteinuria and CKD.

One experimental drug, pirfenidone, has been shown to have beneficial effect on chronic kidney disease. An open-label trial on progressive CKD was carried out, and the data collected from 18 patients who completed a median of 13-month of pirfenidone treatment showed significant improvements in kidney function (the monthly deterioration in GFR slowed from a median of −0.61 ml/min per 1.73 m² during the baseline period to −0.45 ml/min per 1.73 m² with pirfenidone therapy). However, it is noteworthy to emphasize that although pirfenidone slows renal function decline in the open-label trial, clinical data show that pirfenidone does not have any effect on proteinuria. (Cho M. E., et al: Pirfenidone slows renal function decline in patients with focal segmental glomerulosclerosis. Clin. J. Am. Soc. Nephrol. 2: 906-913, 2007). This is in contrast to data which show that pirfenidone caused a decline in urinary protein excretion in a rat model of nephritis (Sabine L. et al: Pirfenidone and candesartan ameliorate morphological damage in mild chronic anti-GBM nephritis in rats, Nephrol. Dial. Transplant 2005, 20: 71-82). There are three more on-going phase II studies on pirfenidone for kidney diseases (http://www.clinicaltrials.gov).

A Chinese patent ZL02114190.8 describes the identification and synthesis of 38 new 5-methyl-1-(substituted phenyl)-2(1H)-pyridone compounds, having the following general structural formula:

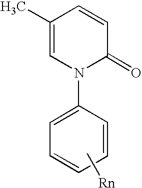

These compounds have been demonstrated to have beneficial effect on fibrosis conditions with much lower toxicity (PCT/CN2006/000651).

Another 5-methyl-1-(substituted phenyl)-2(1H)-pyridone, 1-(3′-fluorophenyl)-5-methyl-2(1H)-pyridone, has been reported to inhibit the proliferation of rat renal fibroblasts in vitro (Tao L. J., et al: J. Cent. South Univ. Med. Sci. 2004, 29:139).

### SUMMARY OF THE INVENTION

The present invention discloses a composition and method for treating proteinuria, which is a major symptom in CKD, and may even be a cause for the disease. It has been found that certain substituted phenyl compounds having structural formula II show ability to reduce proteinuria. Therefore, these compounds may be useful for treating or preventing chronic kidney disease.

These and other features and advantages of this invention will be evident from the following detailed description of preferred embodiments when read in conjunction with the accompanying drawings.

### BRIEF DESCRIPTION OF THE DRAWINGS

FIG. 1 shows the effect of AKF-PD and PDF on p38 activation. Mouse macrophage RAW264.7 cells were pretreated with AKF-PD (100 and 500 μg/ml, 4 h) or PFD (100 and 500 μg/ml, 4 h) and then exposed to LPS (10 ng/ml, 30 min). Total cell lysates were processed by SDS-PAGE and membranes blotted with a phosphospecific p38 and normalized with p38.

FIG. 2 shows the inhibition of TNF-α mRNA synthesis by AKF-PD. Mouse macrophage RAW264.7 cells were pre-treated with AKF-PD or PFD for 4 hours at the following doses: 0, 10 μg/ml, 100 μg/ml, and 500 μg/ml. Cells are then stimulated by treatment with LPS at 10 ng/ml for 20 hours. Expression of TNF-α mRNA is assayed by a quantitative real time PCR analysis using the TaqMan PCR core reagent kit (Applied Biosystems). TNF-α gene expression in the control non-treated cells was taken as 1 and regarded as control. All the other values were expressed as fold increase of the control. The mouse beta actin gene was used as endogenous control. Each column with error bar represents the mean mRNA level±SD of duplicate samples. Statistical comparisons for all three AKF-PD doses relative to control indicate p<0.01; PFD at the doses of 100 and 500 μg/ml relative to control indicate P<0.01.

FIG. 3 shows the inhibition of TNF-α protein expression by AKF-PD. RAW264.7 cells were pre-treated with AKF-PD for 4 hours at the following doses: 0, 30 μg/ml, 100 μg/ml, and 300 μg/ml. Cells were then stimulated by the treatment with LPS at 100 ng/ml for 8 hours. Cell lysate was prepared from the harvested cells. Media from the cell culture were collected, concentrated 10-fold. Both cell lysate (B) and media (A) were subjected to ELISA assay (OptEIA™) specific for mouse TNF-α according to the manufacturer's instructions (BD Biosciences). Each column with error bar represents the average of triplicate TNF-α assay results and the standard deviation (SD). Statistical comparisons for AKF-PD treated cells relative to the controls indicate P<0.001 for secreted levels (A) at all three doses; and P<0.01 for cellular levels (B) at doses of 100 and 300 μg/ml, respectively.

FIG. 4 shows the inhibition of TGF-β production in kidney of rat UUO model. A. Western analysis of TGF-β protein expression. Two rats were sacrificed from groups of control sham operated, UUO, and UUO+AKF-PD at day 7 and day 15. Total protein was extracted from the kidney samples and subjected to Western analysis of TGF-β. The protein loading was normalized with actin. Lane 1, control sham at day 7; lane 2, control sham at day 15; lane 3, UUO at day 7; lane 4, UUO+AKF-PD at day 7; lane 5, UUO at day 15; lane 6, UUO+AKF-PD at day 15. B. Comparison of the intensity of bands on a Western blot by densitometry. The bands were scanned by densitometer and expressed as a fold increase over control sham operated rat at day 7, which was taken as 1.

### DETAILED DESCRIPTION OF THE INVENTION

The present invention discloses evidence that 5-methyl-1-(substituted phenyl)-2(1H)-pyridones can be used to treat proteinuria, particularly proteinuria in chronic kidney diseases. Said pyridones can also be used to treat kidney diseases, including diabetic renal diseases and non-diabetic renal diseases. Kidney diseases include diabetic nephropathy, hypertensive nephropathy, autoimmune glomerular diseases, infection-related glomerular disease, scleorotic glomerular diseases, HIV associated nephropathy, renal amyloidosis, idiopathic glomerular diseases, transplant related kidney fibrosis, and paraneoplastic nephropathy.

Examples of 5-methyl-1-(substituted phenyl)-2(1H)-pyridone having a structural formula of:

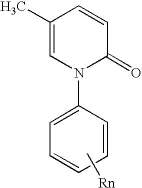

include, but are not limited to, the following compounds:

When n=1 and R═Br, the compounds can be 1-(2′-bromophenyl)-5-methyl-2(1H)-pyridone, 1-(3′-bromophenyl)-5-methyl-2(1H)-pyridone, or 1-(4′-bromophenyl)-5-methyl-2(1H)-pyridone.

When n=1 and R═F, the compounds can be 1-(2′-fluorophenyl)-5-methyl-2(1H)-pyridone, 1-(3′-fluorophenyl)-5-methyl-2(1H)-pyridone, or 1-(4′-fluorophenyl)-5-methyl-2(1H)-pyridone.

When n=1 and R═I, the compounds can be 1-(2′-iodophenyl)-5-methyl-2(1H)-pyridone, 1-(3′-iodophenyl)-5-methyl-2(1H)-pyridone, or 1-(4′-iodophenyl)-5-methyl-2(1H)-pyridone.

When n=2 and R═F, Br, or Cl, the compounds can be 1-(2′,3′-dibromophenyl)-5-methyl-2(1H)-pyridone, 1-(2′,4′-dibromophenyl)-5-methyl-2(1H)-pyridone, 1-(2′,5′-dibromophenyl)-5-methyl-2(1H)-pyridone, 1-(2′,6′-dibromophenyl)-5-methyl-2(1H)-pyridone, 1-(3′,4′-dibromophenyl)-5-methyl-2(1H)-pyridone, 1-(3′,5′-dibromophenyl)-5-methyl-2(1H)-pyridone, 1-(2′,3′-dichlorophenyl)-5-methyl-2(1H)-pyridone, 1-(2′,4′-dichloro-phenyl)-5-methyl-2(1H)-pyridone, 1-(2′,5′-dichlorophenyl)-5-methyl-2(1H)-pyridone, 1-(2′,6′-dichlorophenyl)-5-methyl-2(1H)-pyridone, 1-(3′,4′-dichlorophenyl)-5-methyl-2(1H)-pyridone, 1-(3′,5′-dichlorophenyl)-5-methyl-2(1H)-pyridone, 1-(2′,3′-difluorophenyl)-5-methyl-2(1H)-pyridone, 1-(2′,4′-difluoro-phenyl)-5-methyl-2(1H)-pyridone, 1-(2′,5′-difluorophenyl)-5-methyl-2(1H)-pyridone, 1-(2′,6′-difluorophenyl)-5-methyl-2(1H)-pyridone, 1-(3′,4′-difluorophenyl)-5-methyl-2(1H)-pyridone, or 1-(3′,5′-difluorophenyl)-5-methyl-2(1H)-pyridone.

When n=1 or 2 and R=trifluoromethyl, the compounds can be 5-methyl-1-(2′-trifluoromethylphenyl)-2(1H)-pyridone, 5-methyl-1-(4′-trifluoromethylphenyl)-2(1H)-pyridone, 1-(2′,3′-bis-trifluoromethylphenyl)-5-methyl-2(1H)-pyridone, 1-(2′,4′-bis-trifluoromethylphenyl)-5-methyl-2(1H)-pyridone, 1-(2′,5′-bis-trifluoromethylphenyl)-5-methyl-2(1H)-pyridone, 1-(2′,6′-bis-trifluoromethylphenyl)-5-methyl-2(1H)-pyridone, 1-(3′,4′-bis-trifluoromethylphenyl)-5-methyl-2(1H)-pyridone, or 1-(3′,5′-bis-trifluoromethylphenyl)-5-methyl-2(1H)-pyridone.

When n=1 or 2 and R=methyl, the compounds can be 5-methyl-1-(2′-methylphenyl)-2(1H)-pyridone, 5-methyl-1-(3′-methylphenyl)-2(1H)-pyridone, 1-(2′,3′-dimethylphenyl)-5-methyl-2(1H)-pyridone, 1-(2′,4′-dimethylphenyl)-5-methyl-2(1H)-pyridone, 1-(2′,5′-dimethylphenyl)-5-methyl-2(1H)-pyridone, 1-(2′,6′-dimethylphenyl)-5-methyl-2(1H)-pyridone, 1-(3′,4′-dimethylphenyl)-5-methyl-2(1H)-pyridone, or 1-(3′,5′-dimethylphenyl)-5-methyl-2(1H)-pyridone.

When n=1 or 2 and R=methoxy, the compounds can be 1-(2′-methoxyphenyl)-5-methyl-2(1H)-pyridone, 1-(3′-methoxy-phenyl)-5-methyl-2(1H)-pyridone, 1-(2′,3′-dimethoxyphenyl)-5-methyl-2(1H)-pyridone, 1-(2′,4′-dimethoxyphenyl)-5-methyl-2(1H)-pyridone, 1-(2′,5′-dimethoxyphenyl)-5-methyl-2(1H)-pyridone, 1-(2′,6′-dimethoxyphenyl)-5-methyl-2(1H)-pyridone, 1-(3′,4′-dimethoxyphenyl)-5-methyl-2(1H)-pyridone, or 1-(3′,5′-dimethoxyphenyl)-5-methyl-2(1H)-pyridone.

The present invention provides a composition comprising one or more 5-methyl-1-(substituted phenyl)-2(1H)-pyridone in an amount effective for reducing proteinuria, particularly proteinuria in patients with chronic kidney disease, whereby preserving kidney functionality, said 5-methyl-1-(substituted phenyl)-2(1H)-pyridone having a structural formula of:

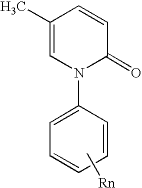

wherein n=1 or 2; R is selected from the group consisting of F, Cl, Br, I, nitro, C₁-C₆ straight-chain alkyl group, C₃-C₆ branched-chain alkyl group, C₁-C₆ straight-chain alkoxy group, C₃-C₆ branched-chain alkoxy group, and halogenated C₁-C₆ alkyl group; and when n=2, not both R are nitro.

The invention also provides the above composition in an amount effective for treating kidney diseases, including diabetic nephropathy, hypertensive nephropathy, autoimmune glomerular diseases, infection-related glomerular disease, sclerotic glomerular diseases, HIV associated nephropathy, renal amyloidosis, idiopathic glomerular diseases, transplant related kidney fibrosis, and paraneoplastic nephropathy.

Examples of 5-methyl-1-(substituted phenyl)-2(1H)-pyridones in the above composition include, but are not limited to, 1-(2′-bromophenyl)-5-methyl-2(1H)-pyridone, 1-(3′-bromo-phenyl)-5-methyl-2(1H)-pyridone, 1-(4′-bromophenyl)-5-methyl-2(1H)-pyridone, 1-(3′-chlorophenyl)-5-methyl-2(1H)-pyridone, 1-(2′-fluorophenyl)-5-methyl-2(1H)-pyridone, 1-(3′-fluorophenyl)-5-methyl-2(1H)-pyridone, 1-(4′-fluorophenyl)-5-methyl-2(1H)-pyridone, 1-(2′-iodophenyl)-5-methyl-2(1H)-pyridone, 1-(3′-iodo-phenyl)-5-methyl-2(1H)-pyridone, 1-(4′-iodophenyl)-5-methyl-2(1H)-pyridone, 1-(2′,3′-dibromophenyl)-5-methyl-2(1H)-pyridone, 1-(2′,4′-dibromophenyl)-5-methyl-2(1H)-pyridone, 1-(2′,5′-dibromophenyl)-5-methyl-2(1H)-pyridone, 1-(2′,6′-dibromophenyl)-5-methyl-2(1H)-pyridone, 1-(3′,4′-dibromophenyl)-5-methyl-2(1H)-pyridone, 1-(3′,5′-dibromophenyl)-5-methyl-2(1H)-pyridone, 1-(2′,3′-dichlorophenyl)-5-methyl-2(1H)-pyridone, 1-(2′,4′-dichlorophenyl)-5-methyl-2(1H)-pyridone, 1-(2′,5′-dichloro-phenyl)-5-methyl-2(1H)-pyridone, 1-(2′,6′-dichlorophenyl)-5-methyl-2(1H)-pyridone, 1-(3′,4′-dichlorophenyl)-5-methyl-2(1H)-pyridone, 1-(3′,5′-dichlorophenyl)-5-methyl-2(1H)-pyridone, 1-(2′,3′-difluorophenyl)-5-methyl-2(1H)-pyridone, 1-(2′,4′-difluorophenyl)-5-methyl-2(1H)-pyridone, 1-(2′,5′-difluoro-phenyl)-5-methyl-2(1H)-pyridone, 1-(2′,6′-difluorophenyl)-5-methyl-2(1H)-pyridone, 1-(3′,4′-difluorophenyl)-5-methyl-2(1H)-pyridone, 1-(3′,5′-difluorophenyl)-5-methyl-2(1H)-pyridone, 5-methyl-1-(2′-trifluoromethylphenyl)-2(1H)-pyridone, 5-methyl-1-(4′-trifluoromethylphenyl)-2(1H)-pyridone, 1-(2′,3′-bis-trifluoromethylphenyl)-5-methyl-2(1H)-pyridone, 1-(2′,4′-bis-trifluoromethylphenyl)-5-methyl-2(1H)-pyridone, 1-(2′,5′-bis-trifluoromethylphenyl)-5-methyl-2(1H)-pyridone, 1-(2′,6′-bis-trifluoromethylphenyl)-5-methyl-2(1H)-pyridone, 1-(3′,4′-bis-trifluoromethylphenyl)-5-methyl-2(1H)-pyridone, or 1-(3′,5′-bis-trifluoromethylphenyl)-5-methyl-2(1H)-pyridone. 5-methyl-1-(2′-methylphenyl)-2(1H)-pyridone, 1-(3′-methylphenyl)-5-methyl-2(1H)-pyridone, 1-(2′,3′-dimethylphenyl)-5-methyl-2(1H)-pyridone, 1-(2′,4′-dimethylphenyl)-5-methyl-2(1H)-pyridone, 1-(2′,5′-dimethylphenyl)-5-methyl-2(1H)-pyridone, 1-(2′,6′-dimethylphenyl)-5-methyl-2(1H)-pyridone, 1-(3′,4′-dimethyl-phenyl)-5-methyl-2(1H)-pyridone, 1-(3′,5′-dimethylphenyl)-5-methyl-2(1H)-pyridone, 1-(2′-methoxyphenyl)-5-methyl-2(1H)-pyridone, 1-(3′-methoxyphenyl)-5-methyl-2(1H)-pyridone, 1-(2′,3′-dimethoxyphenyl)-5-methyl-2(1H)-pyridone, 1-(2′,4′-dimethoxyphenyl)-5-methyl-2(1H)-pyridone, 1-(2′,5′-dimethoxy-phenyl)-5-methyl-2(1H)-pyridone, 1-(2′,6′-dimethoxyphenyl)-5-methyl-2(1H)-pyridone, 1-(3′,4′-dimethoxyphenyl)-5-methyl-2(1H)-pyridone, and 1-(3′,5′-dimethoxyphenyl)-5-methyl-2(1H)-pyridone.

The present invention also provides a pharmaceutical composition comprising the composition described above and a pharmaceutically acceptable carrier. The pharmaceutical composition can be formulated as solution, tablet, capsule, suppository, inhaler, suspension, gel, cream, or ointment.

The present invention provides a method of treating proteinuria in a subject, comprising administering to the subject a composition in an amount effective for treating proteinuria in said subject, wherein the composition comprises one or more 5-methyl-1-(substituted phenyl)-2(1H)-pyridones having a formula of:

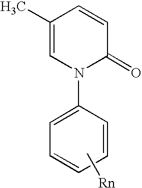

wherein n=1 or 2; R is selected from the group consisting of F, Cl, Br, I, nitro, C₁-C₆ straight-chain alkyl group, C₃-C₆ branched-chain alkyl group, C₁-C₆ straight-chain alkoxy group, C₃-C₆ branched-chain alkoxy group, and halogenated C₁-C₆ alkyl group; and when n=2, not both R are nitro.

In one embodiment of the method, the proteinuria is manifested in chronic kidney diseases. Examples of amounts effective for treating proteinuria include a daily dosage of about 25 mg to about 6,000 mg, a daily dosage of about 50 mg to about 2000 mg, or a daily dosage of about 100 mg to about 1000 mg. These dose ranges are examples only. It is the intent of this invention to cover every single increment in said ranges.

In one embodiment of the method, 1-(3′-fluorophenyl)-5-methyl-2(1H)-pyridone is administered to the subject in need of said treatment. In another embodiment of the method, the composition is administered to the subject as soon as microalbuminuria is confirmed in said subject.

The present invention further provides a method of treating kidney disease in a subject, comprising administrating to the subject a composition in an amount effective for treating kidney disease in said subject, wherein the composition comprises one or more 5-methyl-1-(substituted phenyl)-2(1H)-pyridones having a formula of:

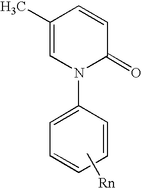

wherein n=1 or 2; R is selected from the group consisting of F, Cl, Br, I, nitro, C₁-C₆ straight-chain alkyl group, C₃-C₆ branched-chain alkyl group, C₁-C₆ straight-chain alkoxy group, C₃-C₆ branched-chain alkoxy group, and halogenated C₁-C₆ alkyl group; and when n=2, not both R are nitro.

Examples of amounts effective for treating kidney disease include a daily dosage of about 25 mg to about 6,000 mg, a daily dosage of about 50 mg to about 2000 mg, or a daily dosage of about 100 mg to about 1000 mg. These dose ranges are examples only. It is the intent of this invention to cover every single increment in said ranges.

In one embodiment of the method, 1-(3′-fluorophenyl)-5-methyl-2(1H)-pyridone is administered to the subject in need of said treatment. In another embodiment of the method, the composition is administered to the subject as soon as microalbuminuria is confirmed in said subject.

The present invention also provides a method of preserving kidney functionality by reducing the degree of proteinuria for progressive kidney disease. Examples of amounts effective for preserving kidney functionality by reducing the degree of proteinuria include a daily dosage of about 25 mg to about 6,000 mg, a daily dosage of about 50 mg to about 2000 mg, or a daily dosage of about 100 mg to about 1000 mg. These dose ranges are examples only. It is the intent of this invention to cover every single increment in said ranges.

In one embodiment of the method, 1-(3′-fluorophenyl)-5-methyl-2(1H)-pyridone is administered to the subject in need of said treatment. In another embodiment of the method, the composition is administered to the subject as soon as microalbuminuria is confirmed in said subject.

The compositions described above can be administered by oral administration, parenteral administration, nasal administration, rectal administration, vaginal administration, ophthalmic application, or topical application.

The invention being generally described will be more readily understood by reference to the following examples which are included merely for purposes of illustration of certain aspects and embodiments of the present invention, and are not intended to limit the invention.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains.

### Example 1

### Inhibition of p38 MAPK Activation by AKF-PD

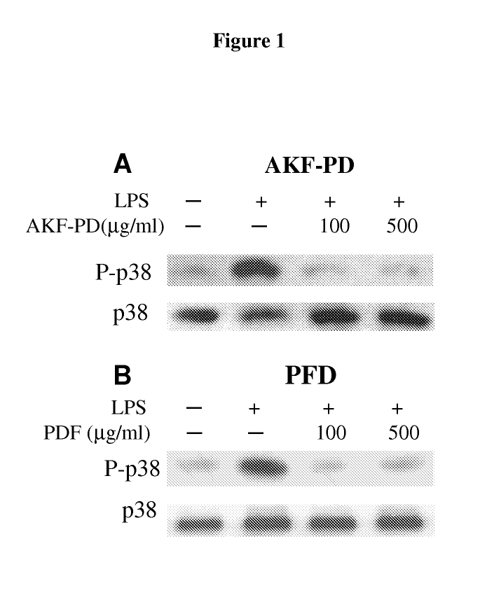

The MAPK family of proteins comprises of three-tiered cascades of Erk1/2, p38, and JNK. The p38 kinase is activated by phosphorylation of a conserved Thr-Gly-Tyr motif in the activation loop. The activated p38 is then able to phosphorylate a wide variety of targets in the cytoplasm and nucleus, resulting in cellular responses such as apoptosis, inflammation, and fibrosis. We first analyzed the anti-inflammatory properties of AKF-PD in p38 signaling in lipopolysaccharide (LPS)-stimulated mouse macrophages RAW264.7 cells. Cells are pre-treated with AKF-PD or pirfenidone (PFD) for 4 hours and then stimulated by treatment with LPS. As shown in FIG. 1, treatment of the cells with LPS resulted in a robotic stimulation of p38 activation. Both AKF-PD (FIG. 1A) and PFD (FIG. 1B) significantly blocked LPS-induced p38 activation. No effects on total p38 protein levels were observed showing that AKF-PD and PFD have no effect on protein stability. The finding that pretreatment with AKF-PD or PFD blocks phosphorylation of the p38 suggests that AKF-PD and PFD might act by inhibiting upstream activators of the p38 pathways. The potential upstream targets for AKF-PD include upstream kinases MAPKK3 (MKK3) and MAPKK6 (MKK6).

### Example 2

### Inhibition of TNF-α mRNA Synthesis in Mouse Macrophage by AKF-PD and PFD

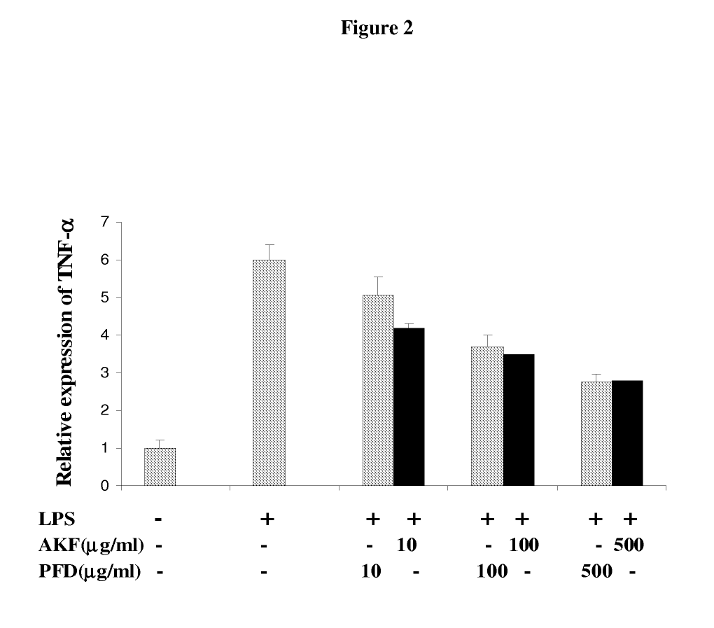

Macrophages are an important source of TNF-α and other pro-inflammatory cytokines during RA and a number of pathways including p38 MAPK have been suggested to contribute to the LPS-induced TNF-α production in this cell type. We next investigated the effect of AKF-PD on TNF-α transcription. Treatment of the cells with LPS significantly increased mRNA level of TNF-α up to 6-fold; both AKF-PD and PFD possessed a dose-dependent inhibition on LPS-stimulated TNF-α mRNA synthesis (FIG. 2). Approximately 50% inhibition was reached at the dose of 500 μg/ml.

### Example 3

### Inhibition of TNF-α Protein Synthesis and Secretion in Macrophage by AKF-PD

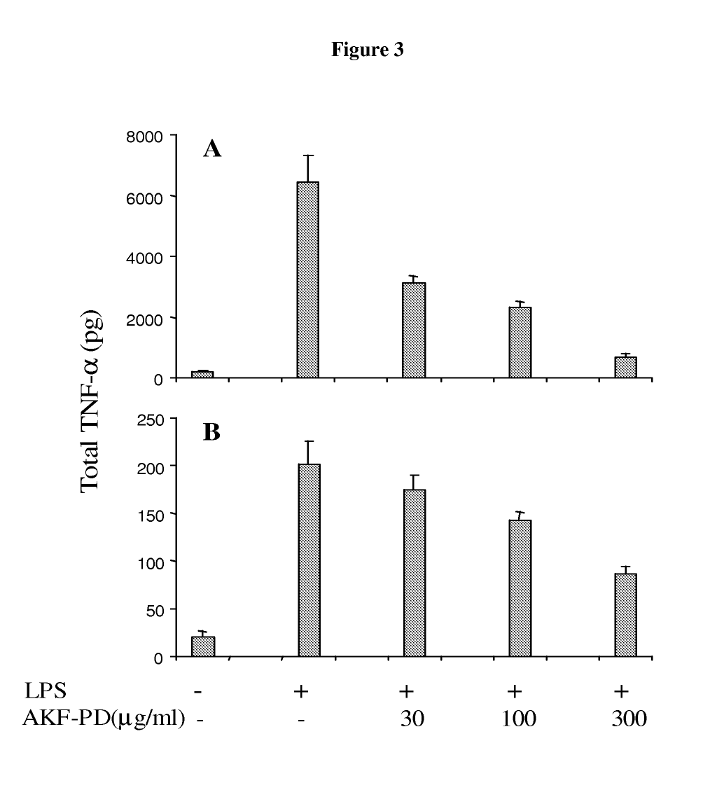

The effect of AKF-PD on TNF-α protein synthesis and secretion was analyzed in LPS-stimulated RAW264.7 cells. Cells were pretreated with AKF-PD and then stimulated by LPS. Both secreted and cellular levels of TNF-α protein were determined. Consistent with the inhibitor effect on mRNA levels, pretreatment of the cells with AKF-PD induced a dose-dependent inhibition on TNF-α protein levels (FIG. 3). AKF-PD, at the dose of 100 μg/ml, caused a 64% inhibition of secreted levels of TNF-α. At the dose of 300 μg/ml, AKF-PD achieved a robust 89% inhibition of secreted levels of TNF-α (FIG. 3A). AKF-PD also suppressed the cellular levels of TNF-α but with less inhibitory effect, achieving approximately 50% inhibition at dose of 300 μg/ml (FIG. 3B). Therefore, the exposure of RAW264.7 cells to AKF-PD exerted a significant and dose-dependent suppression of levels of both cell-associated and secreted TNF-α protein.

### Example 4

### 1-(3′-Fluorophenyl)-5-Methyl-2(1H)-Pyridone (AKF-PD) decreases proteinuria in rats with diabetic nephropathy

Diabetic kidney disease in the rat was induced by streptozotocin (STZ) to test the effectiveness of 5-methyl-2(1H)-pyridone (AKF-PD) in preserving kidney function by reducing the level of proteinuria.

Diabetic nephropathy was induced in Sprague-Dawley rats with a single intravenous injection of STZ (45 mg/kg). Streptozotocin was injected in a single dose of 45 mg/kg, i.v., to rats (Male Sprague-Dawley rats (200-250 g). Age-matched control rats received citrate buffer and were used in parallel with diabetic animals. Two days after STZ injection, plasma glucose levels were measured. Rats having plasma glucose levels>250 mg/dL after 4 weeks were selected for the study. Control and diabetic rats were divided into two groups, consisting of 6 animals each. AKF-PD was suspended in 0.5% sodium carboxy methylcellulose (CMC). From 4 to 8 weeks after the initial STZ injection, the first group of control animals received vehicle, and the second group received a suspension of AKF-PD (500 mg/kg per day) orally. Similarly, one group of diabetic rats received vehicle and the second group received AKF-PD (500 mg/kg per day). At the end of 8th week, rats were kept individually in metabolic cages for 24 h to collect urine for the measurements. Renal function was assessed by measuring plasma and urine levels of creatinine and urea and urine albumin excretion. Plasma glucose levels were also measured at 4 weeks and at the end of the experiment to investigate the effect of AKF-PD on glucose levels. Urinary proteins were measured by Bio-Rad protein assay (Bio-Rad, Hercules, Calif.). Blood urea nitrogen and creatinine levels were measured using Beckman analyzers (BUN Analyzer II, and Creatinine Analyzer II Beckman Instruments Inc., Brea, Calif.).

Four weeks after STZ injection, diabetic animals exhibited increased blood glucose levels (107.82±4.11 and 465.37±14.27 mg/dL for control and diabetic rats, respectively; P<0.05) and decreased bodyweight (250.8±3.06 and 196.72±2.47 g for control and DN rats, respectively; P<0.05) compared with control rats. At the end of 8 weeks, diabetic rats exhibited significantly increased plasma glucose levels and decreased bodyweight compared with control rats. The administration of AKF-PD (500 mg/kg per day) from 4 to 8 weeks did not alter plasma glucose levels and bodyweight in either control or diabetic rats (Table 1). Streptozotocin-injected rats showed significant increases in blood glucose, polyuria, proteinuria and a decrease in bodyweight compared with age-matched control rats. After 8 weeks, diabetic rats exhibited renal dysfunction, as evidenced by reduced creatinine and urea clearance, and proteinuria. Treatment with AKF-PD significantly attenuated renal dysfunction in diabetic rats.

TABLE 1
Effect of 4 weeks treatment with AKF-PD (500 mg/kg per day,
p.o.) on control and streptozotocin-induced diabetic rats

| | Control | Control + AKF-PD | DN | DN + AKF-PD |
|---|---|---|---|---|
| Plasma glucose | 118.60 ± 6.15 | 117.00 ± 6.10 | 445.00 ± 8.75* | 440.25 ± 9.55* |
| levels (mg/dL) | | | | |
| Bodyweight (g) | 243.00 ± 6.60 | 240.35 ± 4.20 | 185.10 ± 3.16* | 192.82 ± 3.91* |
| Urine output (mL) | 10.65 ± 0.06 | 11.15 ± 0.75 | 26.10 ± 1.78* | 15.25 ± 1.75** |
| Urinary albumin | 3.35 ± 0.45 | 3.26 ± 0.25 | 12.35 ± 0.45* | 6.10 ± 0.23** |
| excretion (mg/dL) | | | | |
| Serum creatinine | 0.39 ± 0.03 | 0.36 ± 0.02 | 1.27 ± 0.17* | 0.75 ± 0.13** |
| (mg/dL) | | | | |
| Blood urea | 14.82 ± 0.89 | 13.58 ± 0.79 | 33.05 ± 1.60* | 18.88 ± 1.50** |
| nitrogen (mg/dL) | | | | |

Data are the mean ± SEM (n = 6).
*P < 0.05 compared with the control group without AKF-PD treatment;
**P < 0.05 compared with the diabetic group without AKF-PD treatment.

### Example 5

### Inhibition of TGF-β Production and Reduction of Kidney Tissue Damage in Unilateral Ureteral Obstruction (UUO) Model by AKF-PD

Kidney damage and fibrosis were induced by Unilateral Ureteral Obstruction in rat. The reno-protective effect of AKF-PD was evaluated in the rat UUO model.

Fixed tissues were dehydrated in graded alcohol and embedded in paraffin. Sections were stained with hematoxylin & eosin (HE) for general histology. Injury score was evaluated. On Day 7, the kidneys of rats subjected to ureteral obstruction developed a conspicuous tubulointerstitial injury characterized by tubular dilatation and atrophy, interstitial inflammation, and a marked interstitial fibrosis. The damage was more prominent on Day 15. Renal injury was ameliorated after treatments with AKF-PD as indicated by the lower tubulointerstitial injury scores and interstitial fibrosis expansion of these groups, with P<0.05 for all comparisons (Table 2).

TABLE 2
Tubulointerstitial injury score (TIS) in
renal tissues of rats in different groups.

TIS

| Group | Day 7 | Day 15 |
|---|---|---|
| Sham | 0.01 ± 0.02 | 0.01 ± 0.01 |
| UUO | 4.75 ± 0.70 * | 6.91 ± 1.10 * |
| AKF-PD | 2.64 ± 0.79 * # | 3.82 ± 0.43 * # |

* Compared with the Sham group, P < 0.05;
# Compared with the UUO group, P < 0.05

Table 2. Comparison of histological scores for interstitial compartment. Eight weeks old male SD rats (180 g ˜220 g) were randomly divided into sham surgical group, disease model UUO group, and AKF-PD (500 mg/kg/d) group. Under aseptic condition, all animals in the groups of disease model and AKF-PD had a surgical procedure for ligation of the left side ureter. The animals of the sham surgical group experienced the same surgical procedure except the ligation step. AKF-PD was administrated by gavage from one day prior to the procedure to 15 days post surgical procedure. Normal saline was administrated in a similar fashion to the rats in the groups of disease model and sham surgical. The animals were sacrificed 15 days after surgical procedure and their left kidney were removed for pathological (HE staining) examination. Histological scoring for interstitial compartment was done according to Radford's method (Predicting renal outcome in IgA nephropathy. J. Am. Soc. Nephrology, 1997; 8:199-207).

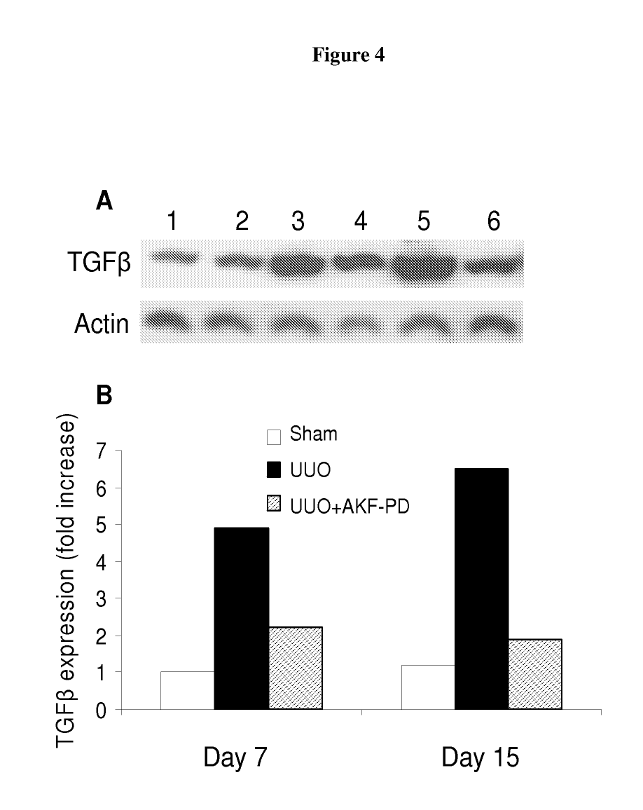

TGF-β is a pro inflammatory cytokine and its overproduction plays critical role in tissue fibrosis. Using UUO model, we analyzed the effect of AKF-PD on TGF-β protein expression by Western blot. As shown in FIG. 4, TGF-β levels increased significantly after 7 and 15 days of renal obstruction (FIG. 4A). Animals treated with AKF-PD during the time period of renal obstruction demonstrated a significant reduction in TGF-β expression. Densitometry analyses of the intensity of the bands indicate that TGF-β protein expression in obstructed kidneys was increased by 4.9- and 6.5-fold than those in the Sham group on Days 7 and 15, respectively. Treatment of UUO rats with AKF-PD significantly reduced TGF-β protein expression by 55% and 71% at Day 7 and 15, respectively (FIG. 4B).

The decreased TGF-β production and reduced tissue injury indicates the possible pharmacological mechanism of AKF-PD for its renoprotective property. Similar to the well established renoprotective property of angiotensin II inhibitors, it is possible that AKF-PD has a complicated pharmacological mechanism for its renoprotective effect.

### Example 6

### AKF-PD: A Novel Anti-proteinuria Therapy for Early Stage Chronic Kidney Disease

The study described below will determine whether the experimental drug AKF-PD can slow the progression of kidney disease in patients with diabetes. Diabetes can damage the kidney and cause proteinuria and eventual kidney failure. AKF-PD has been shown to reduce proteinuria in experimental models of renal disease. In animal models of renal diseases, AKF-PD also benefits glomerulosclerosis and interstitial fibrosis. It is anticipated that AKF-PD may be able to slow the progression of diabetic kidney disease and prolong kidney function.

We will enroll 30 adult patients with type 1 or 2 diabetes with glomerular filtration rate (GFR) between 20-75 ml/min/1.73 m², greater than 300 mg/d of proteinuria, and blood pressure less than or equal to 140/90 on an ACE inhibitor or an ARB (Angiotensin receptor blocker). Participants are randomly assigned to take either 1200 mg of AKF-PD, 2400 mg of AKF-PD, or a placebo by mouth three times a day for 1 year. They return to the clinic 2 weeks after the initial screening visit and then every 3 months throughout the study for fasting blood and urine tests, blood pressure measurement and reviews of any health-related issues. Additional blood samples may be drawn to see if AKF-PD is affecting the level of certain proteins or other related molecules that are thought to be related to kidney disease progression in diabetes.

Patients are asked to check their blood pressure at home at least 3 times a week and record it in a log. A patient whose blood pressure is greater than 130/80 must call the doctor to adjust his or her medications. Patients may also need to monitor their blood sugar more frequently than usual (up to 4 times a day) and possibly give more frequent insulin injections to achieve good control of their diabetes.

Patients are asked to collect 24-hour urine five times during the study: at baseline, 2 weeks, 6 months, 12 months, and 54 weeks (end of study).

Patients will be maintained on the current standard of care for diabetic nephropathy, including an angiotensin converting enzyme (ACE) inhibitor and/or angiotensin receptor blocker (ARB), antihypertensive therapy with blood pressure target of less than 130/80, and aggressive glycemic control with target hemoglobin A1C of less than 7%.

The endpoints will include the percent change in urine albumin excretion and in renal function from baseline to the end of the study period and the levels of urine and plasma TGF-β from baseline to the end of the study period. Renal function will be assessed by the GFR.

Based on data from experimental animal models, it is anticipated that AKF-PD will significantly reduce proteinuria and TGF-β levels to improve renal function and slow the progression of renal disease.

What is claimed is:

1. A method of treating proteinuria in a subject, comprising administering to the subject a composition in an amount effective for treating proteinuria in said subject, wherein the composition comprises one or more 5-methyl-1-(substituted phenyl)-2(1H)-pyridones having a formula of: 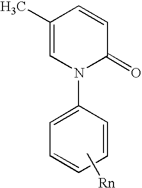
wherein n=1 or 2; R is selected from the group consisting of F, Cl, Br, I, nitro, C₁-C₆ straight-chain alkyl group, C₃-C₆ branched-chain alkyl group, C₁-C₆ straight-chain alkoxy group, C₃-C₆ branched-chain alkoxy group, and halogenated C₁-C₆ alkyl group; and when n=2, not both R are nitro.

2. The method of claim 1, wherein the 5-methyl-1-(substituted phenyl)-2(1H)-pyridone is selected from the group consisting of 1-(2′-bromophenyl)-5-methyl-2(1H)-pyridone, 1-(3′-bromophenyl)-5-methyl-2(1H)-pyridone, 1-(4′-bromophenyl)-5-methyl-2(1H)-pyridone, 1-(2′-chlorophenyl)-5-methyl-2(1H)-pyridone 1-(3′-chlorophenyl)-5-methyl-2(1H)-pyridone, 1-(2′-fluorophenyl)-5-methyl-2(1H)-pyridone, 1-(3′-fluorophenyl)-5-methyl-2(1H)-pyridone, 1-(4′-fluorophenyl)-5-methyl-2(1H)-pyridone, 1-(2′-iodophenyl)-5-methyl-2(1H)-pyridone, 1-(3′-iodophenyl)-5-methyl-2(1H)-pyridone, 1-(4′-iodophenyl)-5-methyl-2(1H)-pyridone, 1-(2′,3′-dibromophenyl)-5-methyl-2(1H)-pyridone, 1-(2′,4′-dibromophenyl)-5-methyl-2(1H)-pyridone, 1-(2′,5′-dibromo-phenyl)-5-methyl-2(1H)-pyridone, 1-(2′,6′-dibromophenyl)-5-methyl-2(1H)-pyridone, 1-(3′,4′-dibromophenyl)-5-methyl-2(1H)-pyridone, 1-(3′,5′-dibromophenyl)-5-methyl-2(1H)-pyridone, 1-(2′,3′-dichlorophenyl)-5-methyl-2(1H)-pyridone, 1-(2′,4′-dichlorophenyl)-5-methyl-2(1H)-pyridone, 1-(2′,5′-dichlorophenyl)-5-methyl-2(1H)-pyridone, 1-(2′,6′-dichloro-phenyl)-5-methyl-2(1H)-pyridone, 1-(3′,4′-dichlorophenyl)-5-methyl-2(1H)-pyridone, 1-(3′,5′-dichlorophenyl)-5-methyl-2(1H)-pyridone, 1-(2′,3′-difluorophenyl)-5-methyl-2(1H)-pyridone, 1-(2′,4′-difluorophenyl)-5-methyl-2(1H)-pyridone, 1-(2′,5′-difluorophenyl)-5-methyl-2(1H)-pyridone, 1-(2′,6′-difluorophenyl)-5-methyl-2(1H)-pyridone, 1-(3′,4′-difluoro-phenyl)-5-methyl-2(1H)-pyridone, 1-(3′,5′-difluorophenyl)-5-methyl-2(1H)-pyridone, 5-methyl-1-(2′-trifluoromethyl-phenyl)-2(1H)-pyridone, 5-methyl-1-(4′-trifluoromethyl-phenyl)-2(1H)-pyridone, 1-(2′,3′-bis-trifluoromethylphenyl)-5-methyl-2(1H)-pyridone, 1-(2′,4′-bis-trifluoromethylphenyl)-5-methyl-2(1H)-pyridone, 1-(2′,5′-bis-trifluoromethylphenyl)-5-methyl-2(1H)-pyridone, 1-(2′,6′-bis-trifluoromethylphenyl)-5-methyl-2(1H)-pyridone, 1-(3′,4′-bis-trifluoromethylphenyl)-5-methyl-2(1H)-pyridone, or 1-(3′,5′-bis-trifluoromethyl-phenyl)-5-methyl-2(1H)-pyridone. 5-methyl-1-(2′-methyl-phenyl)-2(1H)-pyridone, 1-(3′-methylphenyl)-5-methyl-2(1H)-pyridone, 1-(2′,3′-dimethylphenyl)-5-methyl-2(1H)-pyridone, 1-(2′,4′-dimethylphenyl)-5-methyl-2(1H)-pyridone, 1-(2′,5′-dimethylphenyl)-5-methyl-2(1H)-pyridone, 1-(2′,6′-dimethyl-phenyl)-5-methyl-2(1H)-pyridone, 1-(3′,4′-dimethylphenyl)-5-methyl-2(1H)-pyridone, 1-(3′,5′-dimethylphenyl)-5-methyl-2(1H)-pyridone, 1-(2′-methoxyphenyl)-5-methyl-2(1H)-pyridone, 1-(3′-methoxyphenyl)-5-methyl-2(1H)-pyridone, 1-(2′,3′-dimethoxyphenyl)-5-methyl-2(1H)-pyridone, 1-(2′,4′-Page 30 of 36 dimethoxyphenyl)-5-methyl-2(1H)-pyridone, 1-(2′,5′-dimethoxyphenyl)-5-methyl-2(1H)-pyridone, 1-(2′,6′-dimethoxyphenyl)-5-methyl-2(1H)-pyridone, 1-(3′,4′-dimethoxyphenyl)-5-methyl-2(1H)-pyridone, and 1-(3′,5′-dimethoxyphenyl)-5-methyl-2(1H)-pyridone.

3. The method of claim 1 wherein said proteinuria is manifested in chronic kidney disease, wherein said chronic kidney disease includes diabetic renal disease and non-diabetic renal disease.

4. The method of claim 3 wherein the chronic kidney disease includes diabetic nephropathy, hypertensive nephropathy, autoimmune glomerular diseases, infection-related glomerular disease, sclerotic glomerular diseases, HIV associated nephropathy, renal amyloidosis, idiopathic glomerular diseases, transplant related kidney fibrosis, and paraneoplastic nephropathy.

5. The method of claim 3, wherein the amount effective for treating proteinuria in diabetic renal disease and non-diabetic renal disease comprises a daily dosage of about 25 mg to about 6,000 mg.

6. The method of claim 3, wherein the amount effective for treating proteinuria in diabetic renal disease and non-diabetic renal disease comprises a daily dosage of about 50 mg to about 2000 mg.

7. The method of claim 3, wherein the amount effective for treating proteinuria in diabetic renal disease and non-diabetic renal disease comprises a daily dosage of about 100 mg to about 1000 mg.

8. The method of claim 3, wherein the composition is administered through a route selected from the group consisting of oral administration, parenteral administration, nasal administration, rectal administration, vaginal administration, ophthalmic application, and topical application.

9. The method of claim 3, wherein the composition is administered to the subject as soon as microalbuminuria is confirmed in said subject.

10. A method for treating kidney disease in a subject, comprising administrating to the subject a composition in an amount effective for treating kidney disease in said subject, wherein the composition comprises one or more 5-methyl-1-(substituted phenyl)-2(1H)-pyridones having a formula of: 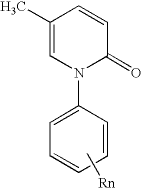
wherein n=1 or 2; R is selected from the group consisting of F, Cl, Br, I, nitro, C₁-C₆ straight-chain alkyl group, C₃-C₆ branched-chain alkyl group, C₁-C₆ straight-chain alkoxy group, C₃-C₆ branched-chain alkoxy group, and halogenated C₁-C₆ alkyl group; and when n=2, not both R are nitro.

11. The method of claim 10, wherein the 5-methyl-1-(substituted phenyl)-2(1H)-pyridone is selected from the group consisting of 1-(2′-bromophenyl)-5-methyl-2(1H)-pyridone, 1-(3′-bromophenyl)-5-methyl-2(1H)-pyridone, 1-(4′-bromo-phenyl)-5-methyl-2(1H)-pyridone, 1-(2′-chlorophenyl)-5-methyl-2(1H)-pyridone 1-(3′-chlorophenyl)-5-methyl-2(1H)-pyridone, 1-(2′-fluorophenyl)-5-methyl-2(1H)-pyridone, 1-(3′-fluorophenyl)-5-methyl-2(1H)-pyridone, 1-(4′-fluoro-phenyl)-5-methyl-2(1H)-pyridone, 1-(2′-iodophenyl)-5-methyl-2(1H)-pyridone, 1-(3′-iodophenyl)-5-methyl-2(1H)-pyridone, 1-(4′-iodophenyl)-5-methyl-2(1H)-pyridone, 1-(2′,3′-dibromophenyl)-5-methyl-2(1H)-pyridone, 1-(2′,4′-dibromophenyl)-5-methyl-2(1H)-pyridone, 1-(2′,5′-dibromo-phenyl)-5-methyl-2(1H)-pyridone, 1-(2′,6′-dibromophenyl)-5-methyl-2(1H)-pyridone, 1-(3′,4′-dibromophenyl)-5-methyl-2(1H)-pyridone, 1-(3′,5′-dibromophenyl)-5-methyl-2(1H)-pyridone, 1-(2′,3′-dichlorophenyl)-5-methyl-2(1H)-pyridone, 1-(2′,4′-dichlorophenyl)-5-methyl-2(1H)-pyridone, 1-(2′,5′-dichlorophenyl)-5-methyl-2(1H)-pyridone, 1-(2′,6′-dichloro-phenyl)-5-methyl-2(1H)-pyridone, 1-(3′,4′-dichlorophenyl)-5-methyl-2(1H)-pyridone, 1-(3′,5′-dichlorophenyl)-5-methyl-2(1H)-pyridone, 1-(2′,3′-difluorophenyl)-5-methyl-2(1H)-pyridone, 1-(2′,4′-difluorophenyl)-5-methyl-2(1H)-pyridone, 1-(2′,5′-difluorophenyl)-5-methyl-2(1H)-pyridone, 1-(2′,6′-difluorophenyl)-5-methyl-2(1H)-pyridone, 1-(3′,4′-difluoro-phenyl)-5-methyl-2(1H)-pyridone, 1-(3′,5′-difluorophenyl)-5-methyl-2(1H)-pyridone, 5-methyl-1-(2′-trifluoromethyl-phenyl)-2(1H)-pyridone, 5-methyl-1-(4′-trifluoromethyl-phenyl)-2(1H)-pyridone, 1-(2′,3′-bis-trifluoromethylphenyl)-5-methyl-2(1H)-pyridone, 1-(2′,4′-bis-trifluoromethylphenyl)-5-methyl-2(1H)-pyridone, 1-(2′,5′-bis-trifluoromethylphenyl)-5-methyl-2(1H)-pyridone, 1-(2′,6′-bis-trifluoromethylphenyl)-5-methyl-2(1H)-pyridone, 1-(3′,4′-bis-trifluoromethylphenyl)-5-methyl-2(1H)-pyridone, or 1-(3′,5′-bis-trifluoromethyl-phenyl)-5-methyl-2(1H)-pyridone. 5-methyl-1-(2′-methyl-phenyl)-2(1H)-pyridone, 1-(3′-methylphenyl)-5-methyl-2(1H)-pyridone, 1-(2′,3′-dimethylphenyl)-5-methyl-2(1H)-pyridone, 1-(2′,4′-dimethylphenyl)-5-methyl-2(1H)-pyridone, 1-(2′,5′-dimethylphenyl)-5-methyl-2(1H)-pyridone, 1-(2′,6′-dimethyl-phenyl)-5-methyl-2(1H)-pyridone, 1-(3′,4′-dimethylphenyl)-5-methyl-2(1H)-pyridone, 1-(3′,5′-dimethylphenyl)-5-methyl-2(1H)-pyridone, 1-(2′-methoxyphenyl)-5-methyl-2(1H)-pyridone, 1-(3′-methoxyphenyl)-5-methyl-2(1H)-pyridone, 1-(2′,3′-dimethoxyphenyl)-5-methyl-2(1H)-pyridone, 1-(2′,4′-dimethoxyphenyl)-5-methyl-2(1H)-pyridone, 1-(2′,5′-dimethoxyphenyl)-5-methyl-2(1H)-pyridone, 1-(2′,6′-dimethoxyphenyl)-5-methyl-2(1H)-pyridone, 1-(3′,4′-dimethoxyphenyl)-5-methyl-2(1H)-pyridone, and 1-(3′,5′-dimethoxyphenyl)-5-methyl-2(1H)-pyridone.

12. The method of claim 10, wherein the kidney disease includes diabetic renal disease and non-diabetic renal disease.

13. The method of claim 10 wherein the kidney disease includes diabetic nephropathy, Hypertensive nephropathy, autoimmune glomerular diseases, infection-related glomerular disease, and sclerotic glomerular diseases, HIV associated nephropathy, renal amyloidosis, idiopathic glomerular diseases, transplant related kidney fibrosis, and paraneoplastic nephropathy.

14. The method of claim 10, wherein the amount effective for treating kidney disease comprises a daily dosage of about 25 mg to about 6,000 mg.

15. The method of claim 10, wherein the amount effective for treating kidney disease comprises a daily dosage of about 50 mg to about 2000 mg.

16. The method of claim 10, wherein the amount effective for treating kidney disease comprises a daily dosage of about 100 mg to about 1000 mg.

17. The method of claim 10, wherein the composition is administered through a route selected from the group consisting of oral administration, parenteral administration, nasal administration, rectal administration, vaginal administration, ophthalmic application, and topical application.

18. The method of claim 10, wherein the composition is administered to the subject as soon as microalbuminuria is confirmed in said subject.
